(19) United States
(12) Patent Application Publication
(10) Pub. No.: US 2011/0160618 A1
(43) Pub. Date: Jun. 30, 2011

(54) APPARATUS FOR THE DIAGNOSIS AND TREATMENT OF BRUXISM

(75) Inventors: Toam Shemesh, Tel Aviv (IL); Ayelet Rubinovitch Trop, Bat-Chen (IL)

(73) Assignee: WISEMED LTD., Migdal HaEmek (IL)

(21) Appl. No.: 13/059,982

(22) PCT Filed: Aug. 6, 2009
(86) PCT No.: PCT/IL2009/000770

§ 371 Date: Feb. 20, 2011

(30) Foreign Application Priority Data
Aug. 25, 2008 (IL) 193669

(51) Int. Cl. A61B 5/103

(52) U.S. Cl. 600/590

(57) ABSTRACT

The present invention is an apparatus for detecting and treating bruxism. The apparatus of the invention comprises a sensing unit, a calculation unit and a stimulation unit. The sensing unit is in charge of measuring the interocclusal distance and reporting this distance to the calculation unit. After receiving the information from the sensing unit, the calculation unit archives and processes the information to determine if the sensing event is a bruxism event. When a sensing event is confirmed as a bruxism event, the calculation unit selects an adequate stimulation and sends a request to the stimulation unit, which applies the selected stimulation. The apparatus of the present invention may be suitable for treating both diurnal and nocturnal bruxism and the intrabuccal components may be removed from the mouth when bruxism events are not expected to occur, i.e. during the day for an individual who suffers only from nocturnal bruxism.

### FIELD OF THE INVENTION

The present invention relates generally to bruxism, and more particularly to an apparatus for the diagnosis and treatment of bruxism.

### BACKGROUND OF THE INVENTION

Bruxism relates to an unconscious oral habit characterized by a rhythmic activity of facial and jaw muscles that causes a forced contact between dental surfaces, accompanied by tooth grinding and clenching. Bruxism can occur during the day, in which case it is known as diurnal bruxism but it occurs generally while the person is asleep. This is called nocturnal bruxism and it represents the third most frequent parasomniac and is the most serious and difficult to treat since the individual is unaware of the grinding behavior. All forms of bruxism entail forceful contact between the biting surfaces of the upper and lower teeth which may cause serious dental damage or discomfort of jaw muscles. The tooth contact in grinding and tapping, involves movement of the lower jaw and audible sounds. Clenching, on the other hand, involves inaudible, sustained, forceful tooth contact but is not accompanied by mandibular movements.

Chronic bruxism may lead to severe tooth problems. Grinding or clenching may cause tooth shortening, cracking or breaking down of the enamel, cusps fractures, reduced facial height, pulpal problems, and hypersensitivity to temperature. Some evidence shows that higher levels of mercury can be found in the blood of some bruxers having mercury based dental fillings. Other symptoms reported are fatigue of facial muscles, jaw pain, osteoartrosis, crepitus, limitation of jaw movements, clicking and locking of the jaw, headaches, neck aches, or earaches. In some cases, bruxism leads to inflammation and blockage of salivary glands due to muscle hypertrophy, thereby blocking the opening of the nearby parotid glands and leading to periodical swelling, pain, inflammation, and abnormal dryness of the mouth. Bruxism may also damage the temporomandibular joints and is therefore believed to be one of the leading causes of temporomandibular disorders. Clenchers destroy their teeth silently, and their habit therefore does not directly affect members of their household. Grinders find themselves in a more uncomfortable position, as many people find the grinding sound unpleasant, irritating, disturbing, or to cause headaches.

A number of different treatments for bruxism have been proposed: relaxation therapies and psychotherapeutic approaches such as massed negative practice (e.g., clenching the teeth while awake to fatigue the jaw muscle); occlusal adjustment (e.g. grinding down of some teeth and artificially restoring others); medication, such as local anesthesia or tranquilizers (U.S. Pat. No. 6,632,843); aversive conditioning (U.S. Pat. No. 6,164,278); usage of interocclusal orthopedic appliances (U.S. Pat. No. 6,302,110); and biofeedback therapies. Biofeedback approaches aim to create awareness signals to treat bruxism and use devices that are placed in the mouth or mounted on the head or the face of the patient. These devices contain various types of sensing elements that report a particular event associated with bruxism and generate a signal causing either a natural reflex to break the grinding behavior or to alarm the patient.

WO 03/059160 discloses, for example, an apparatus mounted on the user's head, temple, forehead or jaw, which comprises biosensors measuring the activity of nerves or muscles related to bruxism, and a drug delivery module, which release a relaxing drug on the specific nerves or muscles. However, one of the main problems with this apparatus is the lack of a controller that can distinguish normal activity of muscles or nerves from an activity associated with bruxism. Furthermore, the use of drugs or anesthetics, which are expensive and may present some side effects, are not particularly recommended in such treatment. Dosage of the drugs should be personalized to avoid incorrect dosage that may be either ineffective or causes undesired somnolence.

U.S. Pat. No. 5,490,520 discloses a dental appliance composed of a mouthpiece incorporating an electrical power source, electrodes and electrical circuitry to provide an electrical stimulus when the upper and lower teeth are in contact, thereby triggering a reflex causing unclenching the teeth.

Similarly, a device based on pressure sensors embedded in a plastic appliance but generating a sound alert is disclosed in U.S. Pat. No. 5,190,051. Mouthpieces, as presented above, are cumbersome devices, complex to manufacture, subjected to dislodgment in the mouth, and usually disturb the good swallowing of saliva.

U.S. Pat. No. 5,586,562 discloses a smaller device, that can be attached to the side of one tooth with the help of release clips. The device has an L-shape and comprises pressure sensors that report contact between the teeth or vibration events that are further treated by a controller and producing an alarming signal to warn the patient if said pressure exceeds a determined threshold. This device is placed in the mouth by engaging the vertically disposed mounting surface on clips hounded on the outer surface of a tooth, therefore placing the horizontally placed biting “element” between two opposing teeth. Even if this device seems more convenient than the other biofeedback devices previously reported in the prior art, it still has some drawbacks. For example, the L-shape of the device results in the sensing part being localized in the interocclusal space, where it may be uncomfortable for the patient. Furthermore, repetitive grinding or clenching may quite rapidly wear down and even destroy the sensor as it is directly in contact with the surfaces exerting the pressure.

Therefore it is an object of the present invention to provide an apparatus that can help sense, treat and eliminate nocturnal and diurnal bruxism.

It is another object of the invention to provide a non-cumbersome apparatus, which has at least one component installed in the mouth of a patient.

It is still another object of the invention to provide an apparatus that can distinguish between bruxism events and normal mandibular movements.

It is still another object of the invention to provide an apparatus that is capable of determining and reporting the intensity of a patient's bruxism habit.

It is still another object of the invention to provide an apparatus that is able to determine and deliver an adequate stimulus to be delivered to the patient, when a bruxism event is detected

Other objectives and advantages of the invention will become apparent from the following description taken in conjunction with the accompanying drawings wherein are set forth, by way of illustration and example, certain embodiments of this invention.

### SUMMARY OF THE INVENTION

The present invention is an apparatus for detecting and treating bruxism comprising three dedicated units, the elements of which are located into one or more components, wherein said units are:
1. a sensing unit adapted to make measurements;
2. a calculation unit adapted to determine distances from said measurements, to detect the possibility of the occurrence of a bruxism event from the value of said distances, to confirm that said possible event is an actual bruxism event that requires treatment, to make logic decisions, and to deliver a signal that a stimulus should be applied to end said confirmed event; and
3. a stimulation unit adapted to receive said signal from said calculation unit and to create and apply said stimulus to end said confirmed bruxism event.

The apparatus of the invention is characterized in that at least some of the elements of said sensing unit are located in a component that is attached to a first tooth located in one jaw of a patient and the measurements made by said sensing unit are indicative of the interocclusal distance between said first tooth and the opposing tooth located in said patient's other jaw.

In one embodiment of the apparatus of the invention, all the elements of the sensing unit, are included in one component, attached to a tooth of a patient. In some embodiments, a passive element is then attached to the opposing tooth in the other jaw. In some other embodiments, the signal emitting element of the sensing unit is located in a first component, which is attached to a first tooth in one jaw of a patient and the detector element of said sensor unit is located in a second component, which is attached to the opposing tooth in the other jaw.

In one embodiment of the apparatus of the invention, the calculation unit comprises a microprocessor, a dedicated software package, memory, two way communication means and an electronic circuit. In one embodiment, all the elements of the calculation unit are included in one single component, said component optionally comprising at least one element of the sensing unit. In another embodiment, the elements of the calculation unit are located in at least two different components.

In a preferred embodiment of the apparatus of the invention, the detection is made by the sensing unit and the calculation unit and the data storage, confirmation and logic decisions are made by the calculation unit. In some embodiments, an interocclusal distance less than a predetermined threshold value is the parameter used by the calculation unit to detect a possible bruxism event. In some other embodiments, an interocclusal distance less than a predetermined threshold value and the duration of a possible bruxism event are the parameters used by the calculation unit to confirm a bruxism event. In still some other embodiments, it is an interocclusal distance less than a predetermined threshold value, the duration of a possible bruxism event, and the interval between consecutive possible bruxism events, are used by the calculation unit to confirm a bruxism event. In still some other embodiments, the calculation unit confirms that, for a given individual, a detected event is a bruxism event based on the bruxism history of said individual, Wherein said history is continually updated by a self learning software program included in the calculation unit.

In a preferred embodiment of the apparatus of the invention, when the calculation unit confirms that a detected event is an actual bruxism event that requires treatment, the calculation unit then decides if a stimulus should be applied, and if so it sends instructions to the stimulation unit to trigger and deliver a stimulus. The stimulation unit comprises one or more of the following:
1. an electro mechanical apparatus that generates vibrations or knocks;
2. means to generate an electrical stimulus;
3. means to generate an audible stimulus;
4. means to generate a visual stimulus;
5. means to generate a olfactory stimulus;
6. means to generate a gustatory stimulus; and
7. means to provide a medication.

In one embodiment, the elements of the stimulation unit are located in one component, said component comprising optionally at least one element of the sensing unit. In some other embodiments, the elements of the stimulation unit are located in at least two different components.

In some embodiments of the apparatus of the present invention, at least one of the components containing elements of the calculation unit and/or the stimulation unit is attached to an external part of the patient or is placed at a location that is in sufficient proximity to the patient so that he will respond to the stimulus. In one embodiment, the type, the duration and the level of the stimulus is predetermined and stored in the calculation unit. In some other embodiments, the type, the duration and the level of the stimulus is selected randomly by the calculation unit. In still other embodiments, the type, the duration and the level of the stimulation is determent logically by the calculation unit.

In a preferred embodiment of the apparatus of the invention, each intrabuccal component is attached to a tooth by one of the following methods: fixed attachment method, fixed detachable attachment method, or detachable attachment, method. The apparatus of the invention may be calibrated by a dentist when it is provided to a patient, or by the patient or any non-professional person by using an external wireless input apparatus.

The calculation unit can be adapted to transmit data and statistical information to a dentist who can use this data and statistical information for diagnostic purposes.

### BRIEF DESCRIPTION OF THE DRAWINGS

The above and other characteristics and advantages of the invention will be more readily apparent through the following examples, and with reference to the appended drawings, wherein:

FIG. 1 shows the different units of the apparatus of the invention and their respective tasks;

FIG. 2 schematically shows one embodiment of the apparatus of the invention, wherein all the units are included into one single intrabuccal component;

FIGS. 3A to 3D schematically show different embodiments of the apparatus of the invention comprising two distinct components, placed internally or externally, and wherein each component comprises one or more elements of the specialized units;

FIGS. 4A to 4F schematically show different embodiments of the apparatus of the invention comprising three distinct components, placed internally or externally, and wherein each component comprises one or more elements of the specialized units;

FIGS. 5A and 5B schematically show different embodiments of the apparatus of the invention comprising four distinct components, placed internally or externally, and wherein each component comprises one or more elements of the specialized units;

FIG. 6A schematically shows one embodiment of the sensing unit of the apparatus of the invention based on distance sensing;

FIG. 6B schematically shows a second embodiment of the sensing unit of the apparatus of the invention based on contact sensing; and

FIG. 7A schematically shows one embodiment of the attaching means used in the fixed detachable attachment method; and

FIG. 7B schematically shows a second embodiment of the attaching means used in the detachable attachment method.

### DETAILED DESCRIPTION OF THE INVENTION

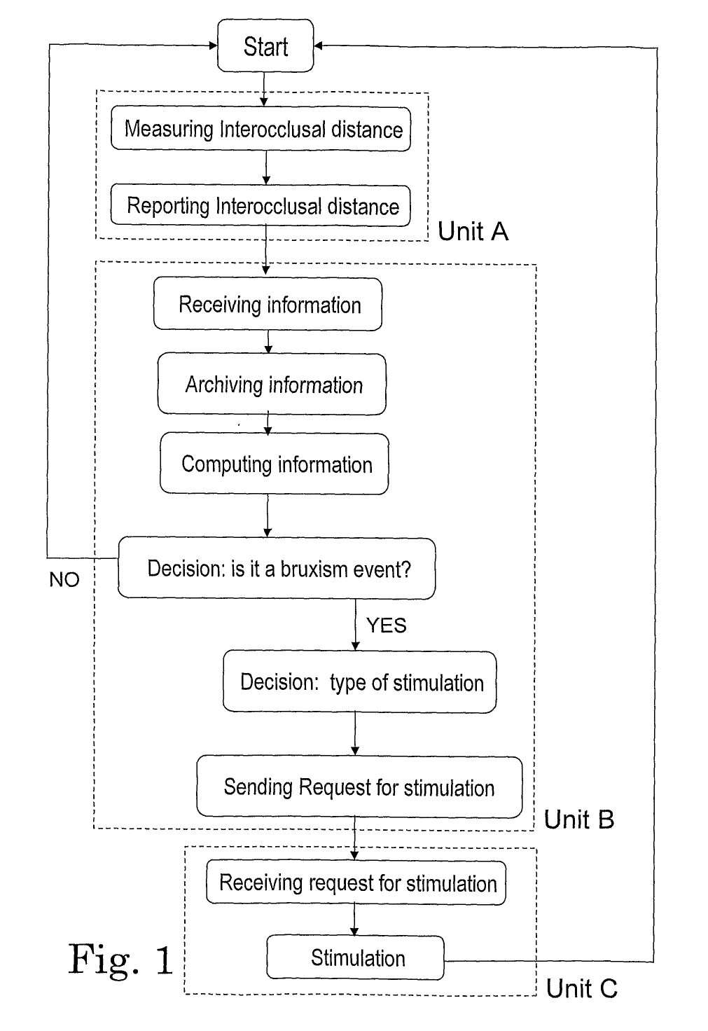

The present invention is an apparatus for detecting and treating bruxism. The apparatus of the invention comprises three units: a sensing unit, a calculation unit and a stimulation unit. As shown in FIG. 1, each unit has dedicated tasks: the sensing unit A is in charge of measuring the interocclusal distance (distance between two opposite teeth which are on the same side of the mouth but on opposite jaws) and reporting this distance to the calculation unit B. After receiving the information from the sensing unit A, the calculation unit B archives and processes the information to determine if the sensing event is a bruxism event. When a sensing event is confirmed as a bruxism event, the calculation unit selects an adequate stimulation and sends a request to the stimulation unit C. After receiving the request from the calculation unit B, the stimulation unit C applies the selected stimulation. The apparatus of the present invention may be suitable for treating both diurnal and nocturnal bruxism and the intrabuccal components may be removed from the mouth when bruxism events are not expected to occur, i.e., during the day for an individual who suffers only from nocturnal bruxism.

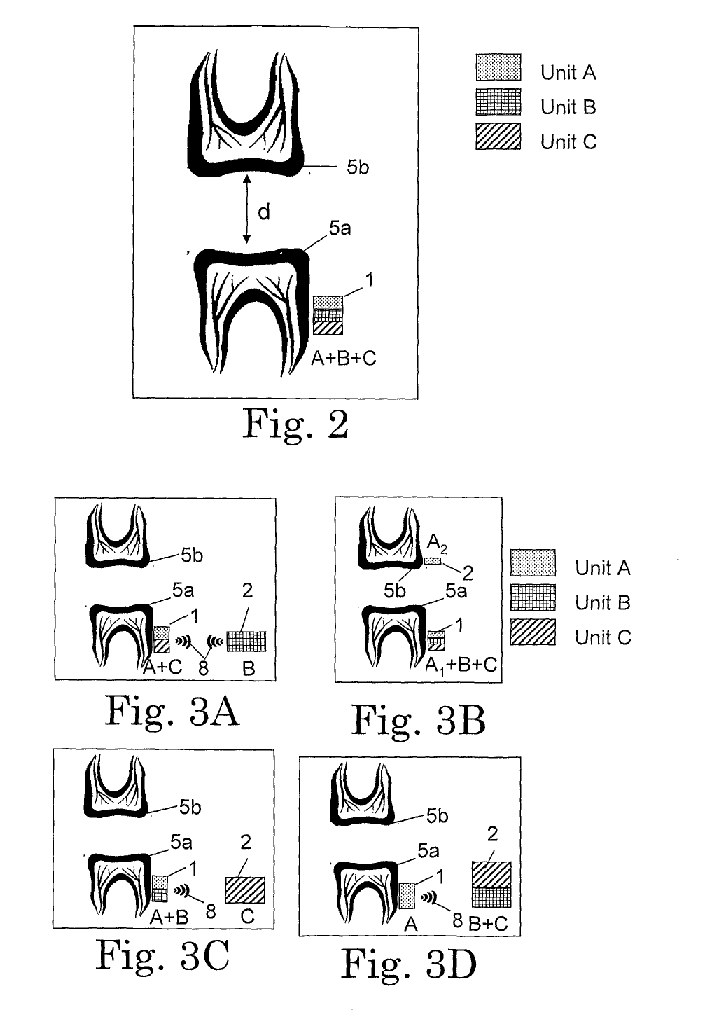

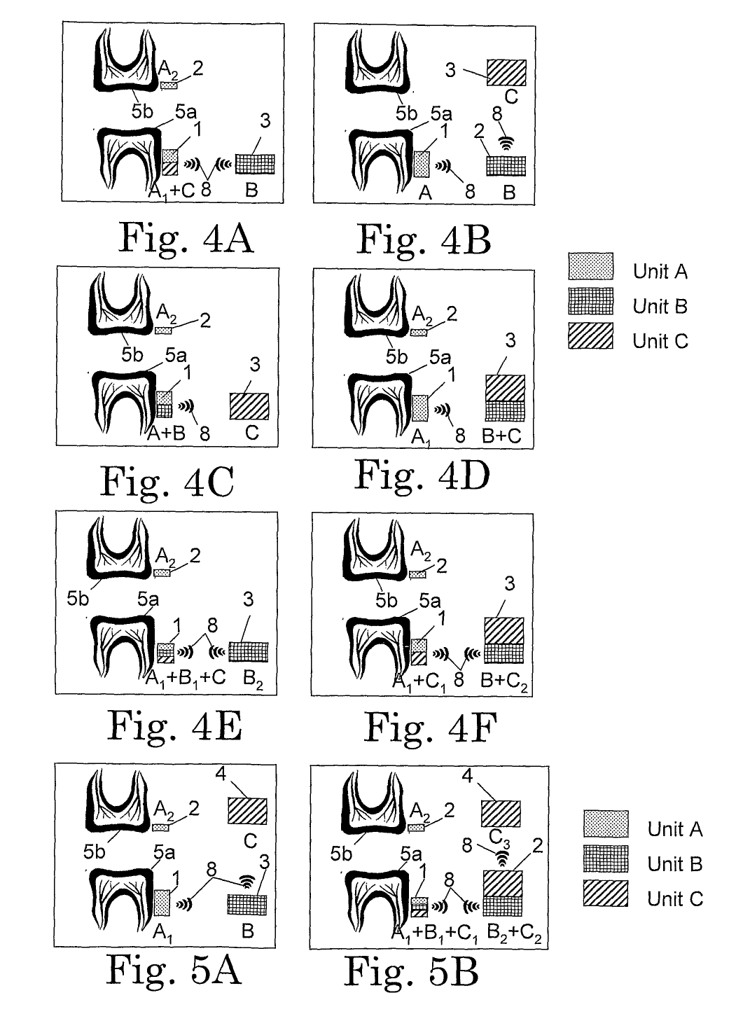

FIGS. 2A-5B show several embodiments of the apparatus of the invention. The elements that comprise the units of the apparatus of the invention may be packaged in one or more packages referred to herein as components. Each component may comprise elements from one or more of the units. At least one component comprising at least one element of the sensor unit must be attached or attachable to a tooth. The other components may be located either inter-orally or extra-orally. Embodiments comprising one (FIG. 2A), two (FIG. 3A-3D), three (FIG. 4A-4F), and four (FIGS. 5A and 5B) components are described herein. In addition to elements of the units, each of the components may comprise its own energy means or the component may be supplied with energy from an external source. In FIGS. 2A-5B, each component (1, 2, 3 and 4) comprises elements of up to three of the different units (A, B or C).

In all embodiments of the apparatus of the invention, the principal task of sensing unit A is to measure the interocclusal distance and report information related to the distance to the calculation unit B, either directly, or via wireless communication means 8. In general sensing unit A comprises two main elements: an emitter that generates a signal of some type and directs it towards an object and a detector that is capable of detecting the signal. In one embodiment of the invention, both main elements of the sensing unit A are located in a single component 1, which is placed on a tooth 5a. A signal is emitted in the direction of the opposite tooth 5b placed in the opposite jaw (FIGS. 2, 3A, 3C, 3D and 4B). The signal is reflected off the opposite tooth and part of the reflected signal is detected in the sensing unit A and used to determine the interocclusal distance d (FIG. 2).

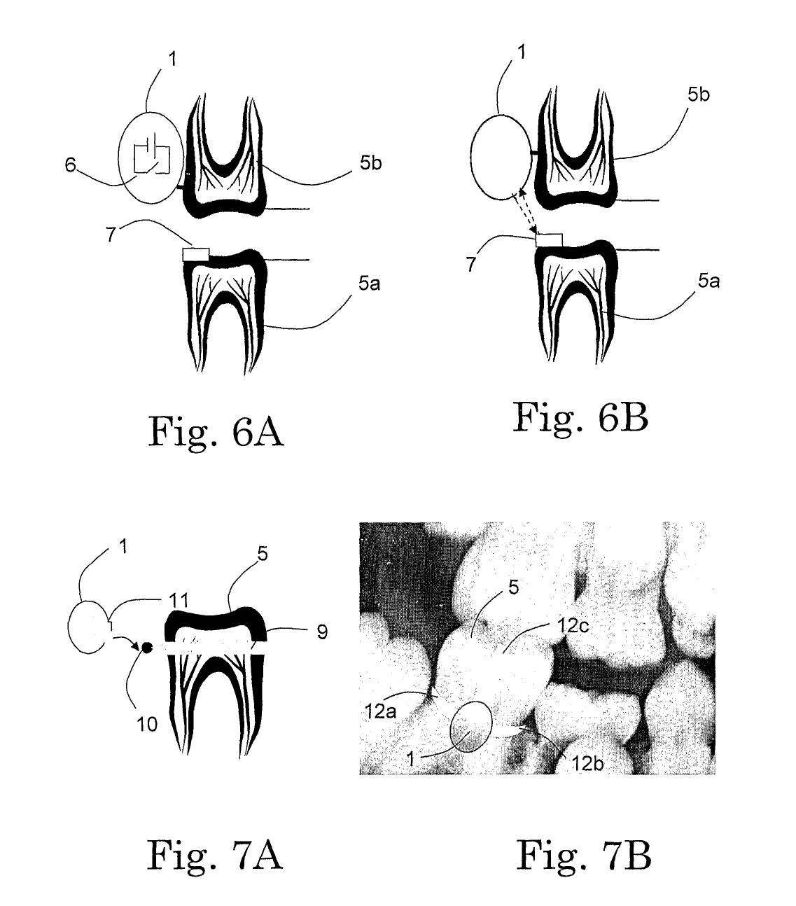

In some embodiments both main elements of the sensing unit, A are located in a single component 1, which is placed on a tooth 5a and a passive element is attached to the tooth opposite the one to which component 1 is attached. The passive element 7 can be for example a metallic piece that closes a switch when there is contact between the two teeth to close an electric circuit 6 located in component 1 (FIG. 6A). In variations of this embodiment, the emitter element sends a signal which is reflected off the passive element 7 and returns to the detector element in component 1 (FIG. 6B). The distance between the two teeth is measured by the strength of the return signal. The passive element 7 can be, for example a metallic piece whose magnetic field can be detected by a suitable detector or a mirror like element for reflecting optical or ultrasonic signals back to the detector. Passive element 7 can either be permanently imbedded in the tooth like a filling (in some cases an existing filling may be used) by a dentist or can be removably attached by any of the methods described herein below.

In other embodiments of the apparatus of the invention, the emitter and detector elements designated A₁ and A₂ of sensing unit A are located in two separate components 1 and 2. In this case, components 1 and 2 are placed on two opposite teeth 5a and 5b of the patient, the component 1 being localized in the lower jaw and component 2 in the upper jaw (or vice versa) (FIGS. 3B, 4A, 4C, 4D, 4E, 4F, 5A, 5B). In some embodiments of the to apparatus of the invention, components 1 and 2, are permanently attached to the teeth of the patient, whereas in other embodiments, one or both components 1 and 2 are releasably attached to the teeth.

As described above, the role of the sensing unit A is to determine the interocclusal distance between the opposing teeth and/or to detect contact, i.e. distance=zero, between them. Close proximity, e.g., a measured distance less than a predetermined threshold value, or contact of the teeth is termed a “sensing event”. When such a sensing event occurs, the software implemented in the calculation unit B interprets the signals and determines if the reported event should be considered as bruxism or as a random event that should be ignored.

The calculation unit B comprises a microprocessor or another type of logic apparatus, a dedicated software package, memory, two way communication means to input instructions and get out results, and an electronic circuit. Data, e.g. bruxism event rate, number of events, and treatment progress, that are collected during the use of the apparatus are accumulated in the memory and may be transmitted to an external apparatus like a PC or a dedicated apparatus which will calculate statistical information and display the collected data and calculated statistical information to the user or authorized health service providers. The dentist can use this data and statistical information for diagnostic purposes. e.g. to decide if treatment is needed, to determine the effectiveness of the current treatment and decide if and what changes are needed, and to determine if treatment can be discontinued.

The calculation unit B can operate with different levels of complexity. In one embodiment of the invention, the calculation unit determines the duration of the sensing event, and if the duration is below a predetermined threshold, the event is considered as a bruxism event. In another embodiment, the duration of the sensing events and the interval between consecutive events are registered and taken into account for the determination of a bruxism event. In another embodiment, the bruxism habits of the patient e.g. frequency and duration of the events, time of day/night, are learnt by a self-learning software program in the calculation unit B, in order to determine the thresholds that will later be used to detect a bruxism event. At this high level of complexity, the calculation unit may decide according to the pattern of previous bruxism events for the specific individual if to generate a stimulus or not.

If a bruxism event is detected and verified by the calculation unit B, then a signal is sent to the stimulation unit C. Stimulation unit C, is preferably located in a component attached to a tooth (FIGS. 2, 3A, 3B, 4A, 4E, 4F, 5B), in which case it administers a mechanical, electrical or soft touch stimulation to the tooth 5, or another oral part, e.g. the inside of the cheek or the gum, of the patient. The stimulation unit C can also be located outside the mouth (FIGS. 3C, 3D, 4B, 4C, 4D, 4F, 5A, 5B), either attached to some external part of the patient, e.g. the wrist or forehead, or at a sufficient proximity to the patient so that he will respond to the stimulus, e.g., on a bedside table next to him. In this case the stimulation unit C is in communication with the calculation unit by means of suitable wireless technologies. In some embodiments, stimulation is based on a mechanical apparatus that generates vibrations or knocks. In one embodiment of the apparatus of the invention, the vibrations are generated by a motor with an eccentric weight. The level of the stimulus is defined by the speed and the duration of the vibration. In another embodiment of the apparatus of the invention, the knock is generated by a piston activated by a an electric solenoid valve. The stimulus level is defined by the speed of the end of the piston hitting the tooth. In other embodiments, the stimulation can be electrical, audible, visible, or affect other senses, e.g. touch, smell, hearing, or taste. In all cases, the icy stimulation triggers a reflex that causes the teeth to involuntarily separate, thereby ending the bruxism event. The decision to apply a stimulus after it is confirmed that the event is a bruxism event is not an automatic one. The software in the calculation unit C might enable other factors, e.g. the time elapsed since the previous stimulus was applied, to be taken into consideration before sending a signal to the stimulation unit.

As in the case of the sensing unit A, the elements of calculation unit B and of stimulus unit C can be distributed amongst one or more components. Examples of some of the possible configurations in which the apparatus of the invention can be implemented are shown schematically in FIGS. 2, 3A-3D, 4A-4F, 5A-5B.

A disadvantage of many apparatus of the prior art is that users become accustomed to the applied stimulus and eventually ignore it completely. To overcome this problem and maintain its effectiveness preferred embodiments of the apparatus of the invention comprise more than one type of stimulus, the type and the level of which can be varied according to a predetermined pattern or randomly in order to avoid the patient's getting accustomed to a specific type of stimulation. The power provided to the different components of the apparatus of the invention can be from an internal battery, that can be replaced or recharged, or can be connected directly to the wall (in the case of an external component).

Each internal component of the invention can be attached to the teeth of a patient in one of three different, ways:
1. Fixed attachment method: In this method the component is attached to the tooth in a way that the patient can not remove it without the intervention of a dentist. For instance, the component is inserted in the tooth in a manner similar to an ordinary filling or is permanently attached to a metal band which is cemented to the tooth.
2. Fixed detachable attachment method: An example of this method is shown in FIG. 7A. According to this method, attachment means 9 are permanently attached to the tooth 5 and the component 1 of the apparatus, is releasably attached to these means as a support. For instance, the attachment means can be implanted in the tooth 5 or permanently attached to a dental crown or to a band which is fitted to the tooth, e.g. an orthodontic band. The attachment means 9 may comprise, e.g., a screw which is screwed into the tooth with a ball shaped head 10 or a hook projecting outwardly from the surface of the tooth. The component is then releasably attached to the attachment means. For example, for the embodiment shown in FIG. 7A, the attachment means may comprise a key hole shaped slot 11 on its outer surface. The large portion of slot 11 is fitted over the round projection 10 and the component 1 is pushed down to attach it to the tooth. The patient can remove the component when not needed for cleaning or recharging. The attachment means 9 may be made of any suitable material such as metal, plastic, or ceramic.
3. Detachable attachment method: an example of this method is shown in FIG. 7B. The component 1 to be attached to the tooth has three flexible arms 12a, 12b and 12c of which two of them 12a and 12b are shaped to fit into interproximal space between the tooth to which the component is to be attached and the two adjacent teeth, thereby “hugging” the tooth. The third arm 12c fits into the occlusal surface like a hook. The component 1 can thus be clipped on the tooth and can be easily released for maintenance or cleaning. In this case no intervention by a dentist is needed to provide attachment of the components. The patient can remove the internal components of the apparatus when not needed, for cleaning, or recharging the battery. The component and the attaching means may be manufactured as a single piece or before use, the apparatus is attached to the attachment means, and then installed on the tooth.

In cases that the apparatus of the invention comprises two internal components, these components may be attached to the teeth using the same method or different ones. e.g. one component can be attached by the fixed attachment method and the other by fixed detachable attachment method.

Calibration of the apparatus of the invention can be performed either by the patient himself or by a dentist, during the installation of the components or during the first use of the apparatus. The calibration of the apparatus comprises defining the parameters of a bruxism event, e.g., distance between the teeth, time of each event, frequency of the events, etc., and defining the different features of the stimulus used when a bruxism event is detected such as the type, the pattern and the intensity. Therefore, the calibration can be clone: i) manually by a dentist when placing the apparatus; ii) by the user himself or any non-professional person according to an installation aid; or electronically by an external wireless apparatus supplied with an instruction guide; or iv) directly from a computer including a dedicated software.

Although embodiments of the invention have been described by way of illustration, it will be understood that the invention may be carried out with many variations, modifications, and adaptations, without exceeding the scope of the claims.

1. An apparatus for detecting and treating bruxism comprising three dedicated units, the elements of which are located into one or more components, wherein said units are:
a) a sensing unit comprised of at least one element adapted to make measurements of distance:
b) a calculation unit adapted to determine distances from said measurements, to detect the possibility of the occurrence of a bruxism event from the value of said distances, to confirm that said possible event is an actual bruxism event that requires treatment, to make logic decisions, and to deliver a signal that a stimulus should be applied to end said confirmed event; and
c) a stimulation unit adapted to receive said signal from said calculation unit and to create and apply said stimulus to end said confirmed bruxism event;
said apparatus characterized in that at least one of said elements of said sensing unit are located in a component that is attached to a first tooth located in one jaw of a patient and the distance measurements made by said sensing unit are indicative of the interocclusal distance between said first tooth and the opposing tooth located in said patient's other jaw.

2. An apparatus according to claim 1, wherein the sensing unit comprises at least one signal emitting element and at least one detector element configured in one of the following ways:
a) one signal emitting element and one detector element are included in one component attached to a tooth of a patient;
b) one signal emitting element and one detector element are included in one component attached to a tooth of a patient and a passive element is attached to the opposing tooth in the other jaw,
c) one of said signal emitting elements is located in a first component, which is attached to a first tooth in one jaw of a patient and one of said detector elements is located in a second component, which is attached to the opposing tooth in the other jaw.

3. (canceled)

4. (canceled)

5. An apparatus according to claim 1, wherein the calculation unit comprises a microprocessor, a dedicated software package, memory, two way communication means and an electronic circuit, wherein the elements of said calculation unit are included in at least one component.

6. (canceled)

7. An apparatus according to claim 5, wherein the elements of the calculation unit are included in one component, which comprises at least one element of the sensing unit.

8. (canceled)

9. (canceled)

10. An apparatus according to claim 1, wherein the calculation unit is adapted to detect a possible bruxism event if an interocclusal distance measured by the sensing unit is less than a predetermined threshold vale.

11. An apparatus according to claim 1, wherein the calculation unit is adapted to confirm that a possible bruxism event is a real bruxism event on the basis of one or more of the following:
a) a measured interocclusal distance and the duration of said possible bruxism event;
b) a measured interocclusal distance, the duration of said possible bruxism event, and the intervals between consecutive possible bruxism events; and
c) for a given individual, the bruxism history of said individual, wherein said history is continually updated by a self learning software program included in said calculation unit.

12. (canceled)

13. (canceled)

14. An apparatus according to claim 1, wherein, the calculation unit is adapted such that after it confirms that a detected event is an actual bruxism event that requires treatment, it can then decide if a stimulus should be applied, and if so it can send instructions to the stimulation unit to trigger and deliver a stimulus.

15. An apparatus according to claim 1, wherein the stimulation unit comprises one or more of the following elements located in at least one component:
a) an electro mechanical apparatus that generates vibrations or knocks;
b) means to generate an electrical stimulus;
c) means to generate an audible stimulus;
d) means to generate a visual stimulus;
e) means to generate a olfactory stimulus;
f) means to generate a gustatory stimulus; and
g) means to provide a medication.

16. (canceled)

17. An apparatus according to claim 15, wherein the elements of the stimulation unit are contained in one component that comprises at least one element of the sensing unit.

18. (canceled)

19. An apparatus according to claim 1, wherein at least one of the components containing elements of the calculation unit and/or the stimulation unit is attached to an external part of the patient or is placed at a location that is in sufficient proximity to the patient so that he will respond to the stimulus.

20. An apparatus according to claim 1, wherein the calculation unit is adapted in at least one of the following ways:
a) to store predetermined information about the type, the duration and the level of the stimulus;
b) to randomly select the type, the duration and the level of the stimulus; and
c) to determine logically the type the duration and the level of the stimulus.

21. (canceled)

22. (canceled)

23. An apparatus according to claim 1, wherein each intrabuccal component is attached to a tooth by one of the following methods: fixed attachment method, fixed detachable attachment method, or detachable attachment method.

24. An apparatus according to claim 1, wherein said apparatus is adapted to be calibrated in at least one of the following ways:
a) by a dentist when said apparatus is provided to a patient; and
b) by the patient or any non-professional person by using an external wired or wireless input apparatus.

25. (canceled)

26. An apparatus according to claim 1, wherein the calculation unit is adapted to transmit data and statistical information to one or both of:
a) a dentist who uses said data and statistical information for diagnostic and calibration purposes; and
b) the patient who uses said data and statistical information for informative or calibration purposes.

27. An apparatus according to claim 1, wherein at least some of the elements of the sensing unit and some of the elements of the calculation unit and some of the elements of the stimulation unit are located in a component that is attached to a first tooth located in one jaw of a patient.
